(19) United States
(12) Patent Application Publication
(10) Pub. No.: US 2004/0067481 A1
(43) Pub. Date: Apr. 8, 2004

(54) Thermal sensor for fluid detection

(76) Inventors: Leslie Leonard, Portola Valley, CA (US); Frederick Stawitcke, Sunnyvale, CA (US); Richard Pittaro, San Carlos, CA (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/363,506

(22) PCT Filed: Jun. 12, 2002
(86) PCT No.: PCT/US02/19052

(51) Int. Cl. C12Q 1/00

(52) U.S. Cl. 435/4

(57) ABSTRACT

Embodiments of the invention provide a method for detecting the arrival of a fluid at or near a thermal sensor. A method includes obtaining a signal from the thermal sensor prior to arrival of the fluid, obtaining another signal during arrival of the fluid, and relating the two signals to indicate the arrival of the fluid. Devices for application of the method are also described.

### TECHNICAL FIELD

In the body, fluidic blood is the result of a dynamic balance between pro-coagulant and anti-coagulation reactions. This is accomplished through the coagulation cascade, which is a series of enzymatic reactions. The endpoint of this enzymatic cascade is the conversion of soluble fibrinogen to insoluble fibrin, which forms the basis of the physical meshwork that prevents the flow of blood. The enzyme that converts fibrinogen to fibrin is thrombin, which itself is generated from a soluble, inactive precursor. The coagulation cascade can be initiated/activated through either the extrinsic or intrinsic pathway. Each pathway begins with a separate set of enzymatic reactions, eventually intersecting in a common pathway through thrombin activation, and leading to the generation of fibrin.

### BACKGROUND ART

In the clinical testing environment, there are a number of available tests to measure the competency of the coagulation system. There are tests for both overall pathway functioning and for isolated individual factors, e.g., ACT, TT, APTT, PT, fibrinogen, Factor V, Factor VIII, etc. The overall capacity of the blood to clot can be measured in terms of “clotting time”, with the rate being measured in seconds. Two common assays used for this are the activated partial thromboplastin time (APTT, measuring intrinsic pathway activation) or prothrombin time (PT, extrinsic pathway activation). These assays are particularly useful for monitoring therapeutic anticoagulation status, i.e., heparin (APTT) or warfarin (PT) anticoagulation.

The majority of commercially available clotting tests measure the endpoint of the cascade, i.e., the formation of the fibrin meshwork. Monitoring this physical change in the blood fluidity can be done in a variety of ways, such as, light scattering, electrical fluid resistance, or physical resistance to movement of magnetic particles added to the blood. More recently, technology has been described which measures the activity of the final enzyme reaction and is correlated to coagulation time. In this technique, the assay is a kinetic measurement of an enzyme reaction, rather than the endpoint assay of physical meshwork.

### DISCLOSURE OF INVENTION

Advantages are achieved by using a method for easily measuring the zero or start-time of an assay and applying such a technology to kinetic assays, where it is critical to have a measurement of the rate of reaction, e.g., other assays that rely upon enzymatic reactions to give a result that can be detected. In addition, advantages are achieved by using a method that is extensible to additional uses and applications, e.g., to provide a means to accurately detect successful sample introduction into a microfluidic cartridge, to detect sample arrival at any predefined point or local environment in a micro-fluidic pathway, to detect sample arrival at a particular sensor or array of sensors, or to perform similar functions for fluids that are not samples containing the analyte to be measured, e.g., subsequent reagents or wash fluids.

Embodiments of the invention generally relate to a method for using a thermal sensor to detect the presence or arrival of a fluid. More particularly, embodiments of the invention relate to the application of enzyme assays where it is important to measure the rate of the reaction. Embodiments of the invention can be particularly useful for detecting the presence of a fluid in a micro-fluidic cartridge.

Embodiments of the invention use the signal obtained from a thermal sensor to define the zero or start time of an assay for which knowing the rate of reaction is important or useful. The same or similar technique can be used to detect the presence of a fluid at a given location in a microfluidic device.

Embodiments of the invention provide a method for reading a signal from a thermal sensor to determine the arrival of a fluid in or near the local environment of the thermal sensor. Generally, a first signal from the thermal sensor is read prior to arrival of the fluid, a second signal is read as the fluid arrives, and the first signal and the second signal are related to provide an indicator that the fluid has arrived. The method may be particularly useful for identifying a zero point in an assay measuring a property of the fluid. In one embodiment, the method comprises determining the temperature of the local environment at the thermal sensor before the fluid arrives, determining the temperature of the local environment at the thermal sensor as the fluid arrives, and calculating the thermal differential, which may be used to indicate the arrival of the fluid. Embodiments of the invention may also be used in an analytical device, which contains a thermal sensor, where the analytical device is used for measuring a property of the fluid.

In another embodiment of the invention, the thermal sensor is used to obtain a signal corresponding to the arrival of a biochemical fluid in the local environment of the thermal sensor, and this arrival is used to define the initiation of a chemical or enzymatic reaction and therefore defines the zero-time for that assay.

In an embodiment of the invention, the thermal sensor is used to obtain a signal to identify the location of a sample or other fluid at any desired and defined point within a microfluidic channel environment.

### BRIEF DESCRIPTION OF DRAWING

The objects, advantages and features of this invention will be more readily appreciated from the following detailed description, when read in conjunction with the accompanying drawing, in which:

FIG. 1 schematically illustrates some possible configurations of thermal sensors as may be used in the present invention.

FIG. 1A illustrates a thermal sensor near a surface over which a fluid may flow. An alternative position for the thermal sensor, exposed to the fluid flowing across the surface, is also illustrated.

FIG. 1B shows a configuration of a thermal sensor with a separate heating element.

FIG. 1C depicts three thermal sensor/heating element pairs located near each other alongside a surface.

FIG. 2 schematically illustrate several positions for the thermal sensors relative to analysis sites.

FIG. 2A illustrates positions for thermal sensors relative to a channel having an analysis site.

FIG. 2B shows possible positions for thermal sensors relative to analysis sites arranged in an array on a surface.

FIG. 3 schematically illustrates a device where a method having features of the invention may be employed. The figure depicts several possible configurations of thermal sensors relative to channels and analysis sites.

### BEST MODE FOR CARRYING OUT THE INVENTION

U.S. Pat. Nos. 5,591,403, 5,110,727, 6,066,504, 5,344,754, 5,049,487, 5,461,910, 6,208,254B1, 6,184,773B1 and U.S. Pat. No. 5,975,485 are hereby incorporated by reference herein in their entirety.

Further aspects and advantages of the invention will be set forth in part in the description which follows, and in part will be obvious from the description, or may become readily apparent through practice of the invention. It is to be understood that both the foregoing general description and the following detailed description are exemplary and explanatory only and are not restrictive of the invention, as claimed. The invention is not limited to specific compositions, process steps, or equipment, as such may vary. It is also understood that the terminology used herein is for the purpose of describing particular embodiments only, and in not intended to be limiting.

It must be noted that, as used in the specification and the appended claims, the singular forms “a”, “an”, and “the”, include plural referents unless the context clearly dictates otherwise. Thus for example, reference to “a thermal sensor” and “an enzymatic assay” or “local environment” can include more than one sensor, assay or environment.

In describing and claiming the present invention, the following terminology will be used in accordance with the definitions set out below.

The term “local environment” refers to the area at, near, next to and adjoining the thermal sensor or detector element being applied in a device or apparatus. Measurements and changes in the local environment are used for determining the arrival or exit of a fluid. Local environments may include micro-fluidic channels, pathways, cartridges, walls, surfaces, fluids, polymer materials, hydrogels, sol-gels, cavities, porous matrixes, or similar devices in or near which a thermal sensor or detector element may be positioned and in which a fluid may be directed toward, away from or by the thermal sensor or detector element.

The term “biomolecular fluid” refers to any fluid that comprises biological fluids and/or biological components (for example, proteins, glycoproteins, DNA, RNA, etc), substances, or materials. Some examples of biological fluids include blood, plasma, serum, buffer matrices containing with proteins or nucleic acids, urine, cerebral spinal fluid, saliva, enzymatic mixtures substances and other related substances and fluids that are well known in the analytical and biomedical art.

A rate-based assay is an assay which measures the rate of change in a property of a sample with respect to time. The “zero- or start-time” refers to the initiation signal for the timing cycle of the rate-based assay. The rate-based assay may result from a reaction such as a chemical or enzymatic reaction which occurs at an analysis site.

The term “analysis site” refers to a location in a device where there is any use, singly or in combination, of chemical test reagents and methods, electrical test circuits and methods, physical test components and methods, optical test components and methods, and biological test reagents and methods to yield information about a biomolecular fluid. Such methods are well known in the art and may be based on teachings of, e.g. Tietz Textbook of Clinical Chemistry, 3d Ed., Sec. V, pp. 776-78 (Burtis & Ashwood, Eds., W. B. Saunders Company, Philadelphia, 1999); U.S. Pat. No. 5,997,817 to Chrismore et al. (Dec. 7, 1999); U.S. Pat. No. 5,059,394 to Phillips et al. (Oct. 22, 1991); U.S. Pat. No. 5,001,054 to Wagner et al. (Mar. 19, 1991); and U.S. Pat. No. 4,392,933 to Nakamura et al. (Jul. 12, 1983), the teachings of which are hereby incorporated by reference in their entirety, as well as others. Analysis sites may include detectors that test electrochemical properties of the biomolecular fluid (e.g. conductivity), or they may include optical means for sensing optical properties of the biomolecular fluid (e.g. chemiluminescence, fluorescence, or dye activation vie enzymatic action), or they may include biochemical reagents (e.g. antibodies, substrates, or enzymes) to sense properties (e.g. presence of antigens, clotting time, or clot lysis) of the biomolecular fluid. The analysis site may comprise biosensing or reagent material that will react with an analyte (e.g. glucose) in the biomolecular fluid so that information about the analyte may be obtained.

The term “thermal differential” refers to a change or difference in temperature. Changes in temperature are usually defined as measuring the temperature at one point in time and then talking a subsequent measurement in the same position at a different point in time. The differential is determined by the measured difference or change from the first reading to the second reading.

The term “thermal sensor” refers to temperature sensing devices, such as thermistors, thermocouples, resistor thermometers, any thermally resistive material, and other temperature sensing devices. In some cases, the thermal sensor may comprise a device which integrally incorporates a separate heating element. As used herein, the term “detector element” refers to a combination of a heating element in close proximity to a temperature sensing device. A “detector element” also includes the special case where the temperature sensing device is the heating element.

The term “thermistor” refers to thermally sensitive resistors which can exhibit predictable and precise changes in electrical resistance when subjected to a corresponding change in temperature.

The term “thermocouple” refers to devices that comprise two metals joined at a junction point which produce a small voltage which varies as a function of temperature at the junction.

The term “signal” when used in relation to a thermal sensor, a heating element, or a detector element refers to a voltage, current, power, electrical resistance, phase shift, or change of voltage, current, power, electrical resistance, or phase shift with respect to time, which can be measured from a thermal sensor, heating element, or detector element. Temperature changes or other thermodynamic changes in the local environment of the thermal sensor are reflected in the signal.

A “dynamic change” is defined as a change in the thermodynamic properties of the local environment as a result of the arrival of a fluid at or near the local environment. The thermodynamic properties affected include the temperature, the thermal conductivity, the thermal mass, and the thermal time constant. Changes in the thermal properties can be detected according to the method of the present invention using a thermal sensor or a detector element.

A constant prescribed parameter is one which is held to a constant value or sequence of values that are held constant during specific phases of operation of the device.

A time-varying prescribed parameter is one which is varied as a known function or waveform in time.

A time-varying measured parameter is one which cannot be usefully described by a single numerical value which represents its “value”. Rather it must be described as a sequence of values, or by a representative mathematical function which varies as a function of time.

Microfluidic devices are generally known as devices which analyze small volumes of sample, such as less than one milliliter of sample, preferably less than 500 microliters, more preferably less than 250 microliters. To perform analyses on such small samples, microfluidic devices also generally have features whose dimensions (such as diameters of channels) are less than 1 millimeter, preferably less than 500 micrometers, more preferably less than 250 micrometers.

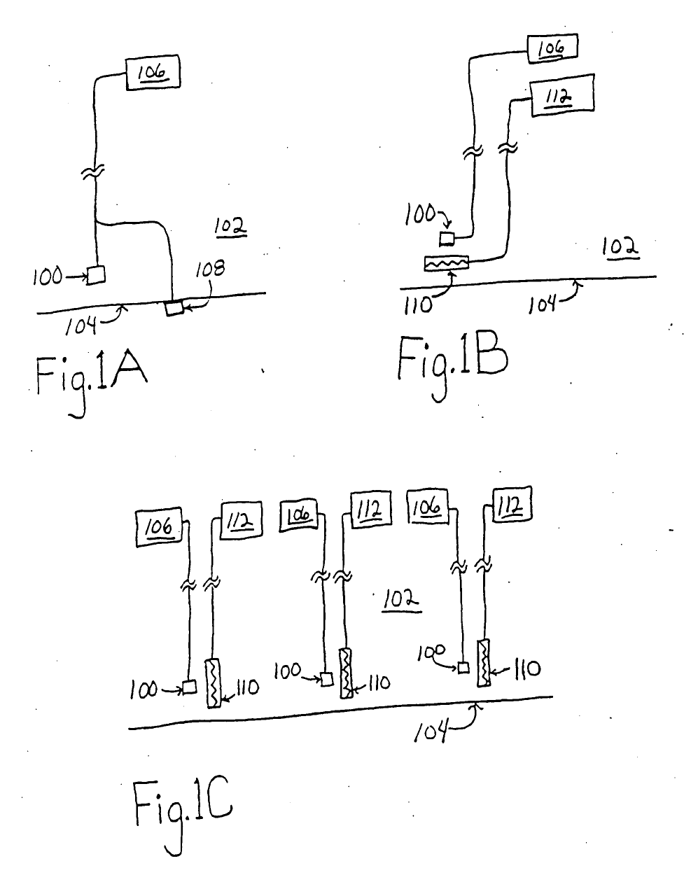

Embodiments of the present invention can be better understood with reference to the figures. In the figures, like numbers generally refer to the same or similar features between the different figures. FIG. 1 schematically illustrates configurations of thermal sensors as may be used in embodiments of the invention. In FIG. 1A, a thermal sensor 100 embedded in a substrate 102 adjacent to a surface 104 over which a fluid may flow. The surface may be, for example, a wall of a channel through which fluid may flow or a surface of a planar device over which fluid may flow. The thermal sensor 100 is in electrical communication with a signal conditioning element 106, which may be embedded in the substrate 102 or may be remotely located. The signal conditioning element 106 receives the signal from the thermal sensor 100 and modifies it by means such as amplifying it and filtering it to reduce noise. FIG. 1A also depicts a thermal sensor 108 located at an alternate location on the surface where it is directly exposed to the fluid flow.

FIG. 1B shows a configuration of a thermal sensor 100 adjacent to a separate heating element 110. The thermal sensor 100 and the heating element 110 are embedded in a substrate 102 adjacent to a surface 104 over which a fluid may flow. In an alternate embodiment, one or more additional thermal sensors may be adjacent the heating element and may provide for increased signal sensitivity. The thermal sensor 100 is in electrical communication with a signal conditioning element 106, which may be embedded in the substrate 102 or may be remotely located. The signal conditioning element 106 receives the signal from the thermal sensor 100 and modifies it by means such as amplifying it and filtering it to reduce noise. The beating element 110 is in electrical communication with a power supply and control element 112, which may be embedded in the substrate 102 or may be remotely located. The power supply and control element 112 provides a controlled source of voltage and current to the heating element 110.

FIG. 1C depicts a configuration of thermal sensors 100 having three thermal sensor/heating element pairs (100/110), or detector elements, (with associated signal conditioning elements 106 and power supply and control elements 112 as described in FIG. 1B) embedded in a substrate 102 near each other alongside a surface 104. The figure depicts the thermal sensors 100 arranged in a linear fashion parallel to the surface 104, but any operable configuration may be used. In alternate embodiments, fewer than three or more than three thermal sensor/heating element pairs (100/110) may be used to indicate the arrival of fluid flowing across a surface 104. In other embodiments, self heating thermal sensors are used, eliminating the separate heating elements.

Embodiments of the present invention provide a simple and accurate methodology for detecting the arrival of a fluid at a defined location. Such detection can be particularly useful to define the zero- or start-time of a timing cycle for measuring rate-based reactions. This can be used in biochemical assays used to detect a variety of analytes present in a variety of types of biological specimens or fluids and for rate-based reactions such as enzymatic reactions. Examples of relevant fluids include, blood, serum, plasma, urine, cerebral spinal fluid, saliva, enzymatic substances and other related substances and fluids that are well known in the analytical and biomedical art. The reaction chemistry for particular assays to analyze biomolecular fluids is generally well-known, and selection of the particular assay used will depend on the biological fluid of interest.

Assays that are relevant to embodiments of the present invention include those that result in the measurement of individual analytes or enzymes, e.g., glucose, lactate, creatinine kinase, etc, as well as those that measure a characteristic of the total sample, for example, clotting time (coagulation) or complement-dependent lysis. Other embodiments for this invention provide for sensing of sample addition to a test article or arrival of the sample at a particular location within that article.

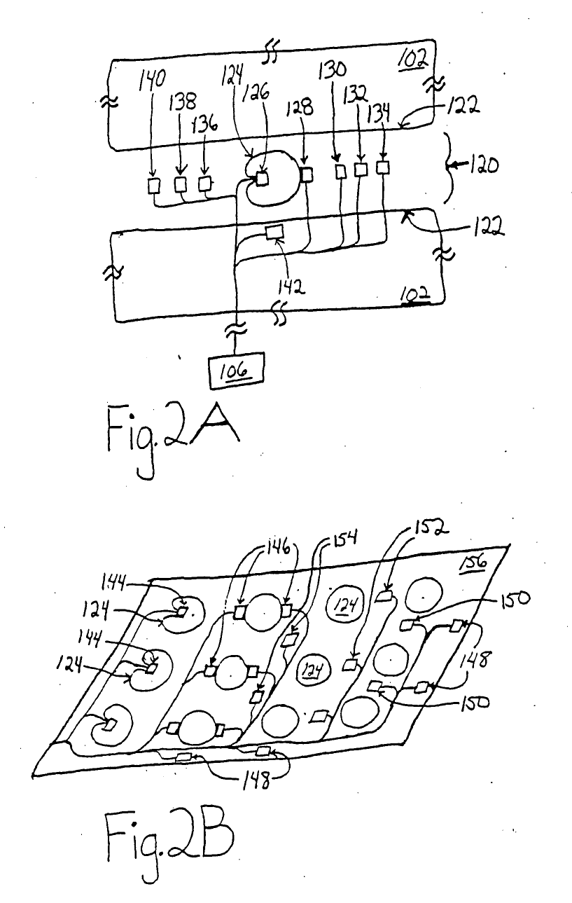

FIG. 2 schematically illustrate several positions for the thermal sensors relative to analysis sites. Referring now to FIG. 2A, a substrate 102 defines a channel 120 having an interior surface 122 over which fluid may flow. An analysis site 124 is located within the channel 120 where fluid flowing in the channel 120 may contact the analysis site 124. In various embodiments, the analysis site 124 may alternatively be upon the interior surface 122, recessed into the substrate 102, or essentially flush with the interior surface 122. FIG. 2A, depicts several possible locations for thermal sensors relative the substrate, the channel, and the analysis site; also, other locations may be useful and will depend upon the design of the device, as will be apparent to those of skill in art. In use, thermal sensors may be omitted from one or more of the locations depicted in FIG. 2A, depending on the intended design. A recess in the analysis site 124 may provide the location for a thermal sensor 126, as may the perimeter of the analysis site provide the location for a thermal sensor 128. One or more thermal sensors 130, 132, 134 may be located on the upstream side of the analysis site 124 (as fluid flows from right to left in FIG. 2A), or one or more thermal sensors 136, 138, 140 may be located on the downstream side of the analysis site 124. The thermal sensor may be embedded in the substrate near the surface, as thermal sensor 142 is depicted. In various other embodiments, the thermal sensor(s) may be located upon the interior surface, recessed into the interior surface, or essentially flush with the interior surface. Each thermal sensor may also be associated with a signal conditioning element, heating element, and power supply and control elements, as described above, and a single signal conditioning element, heating element, or power supply and control element may be associated with more than one thermal sensor.

FIG. 2B shows possible positions for thermal sensors relative to analysis sites 124 arranged in an array on a surface 156. A recess in the analysis site 124 may provide the location for a thermal sensor 144, as may the perimeter of the analysis site provide the location for a thermal sensor 146. The edge of the surface surrounding the array of analysis sites may provide the position for one or more thermal sensors 148. Thermal sensors may be positioned between analysis sites in a particular row 150 or column 152 of the array, or may be arranged on the diagonal 154. In various embodiments, the thermal sensor(s) may be may be embedded in the substrate near the surface or may be located upon the surface, recessed into the surface, or essentially flush with the surface. Each thermal sensor may also be associated with a signal conditioning elements, heating elements, and power supply and control elements, as described above, and a single signal conditioning element, heating element, or power supply and control element may be associated with more than one thermal sensor.

The use of small thermal sensors can be useful in miniaturized systems, such as microfluidic devices, which perform biomolecular analyses on very small fluid samples. Such analyses generally include passing a biomolecular fluid through, over, or adjacent to an analysis site and result in information about the biomolecular fluid being obtained through the use of reagents and/or test circuits and/or components associated with the analysis site.

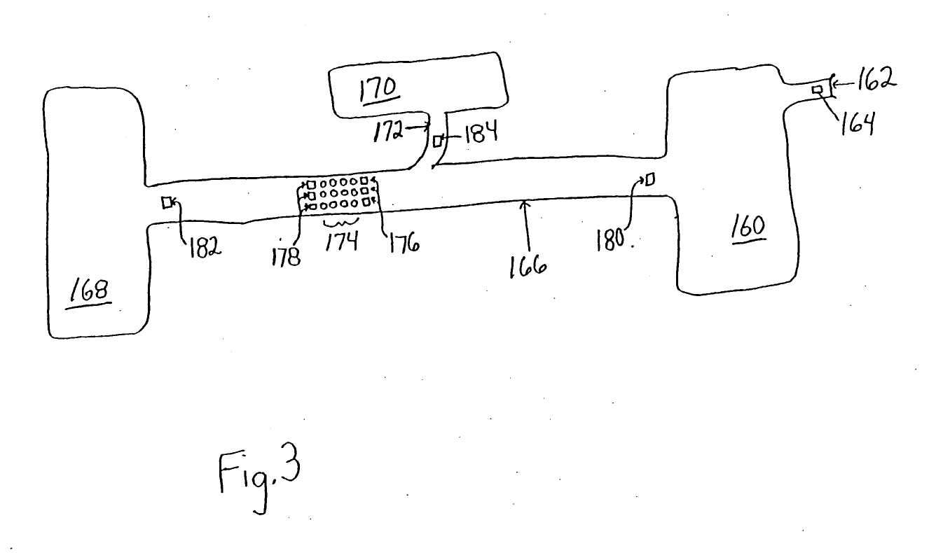

FIG. 3 depicts several possible configurations of thermal sensors relative to channels and analysis sites. The device schematically depicted in FIG. 3 may be, e.g., a microfluidic device for analyzing a small volume of a sample fluid, e.g. a biomolecular fluid. The device has a sample reservoir 160 for holding a quantity of a sample fluid. The sample fluid is introduced to the sample reservoir 160 via a sample inlet port 162 in fluid communication with the sample reservoir 160. A thermal sensor 164 is located in or near the sample inlet port 162. A primary channel 166 originates at the sample reservoir 160 and terminates at an outflow reservoir 168. One or more supplemental reservoirs 170 are optionally present and are in fluid communication with the primary channel 166 via one or more supplemental channels 172, which lead from the supplemental reservoir 170 to the primary channel 166. The supplemental reservoir 170 functions to hold fluids necessary for the operation of the assay, such as reagent solutions, wash solutions, developer solutions, fixative solutions, et cetera. In the primary channel 166 at a predetermined distance from the sample reservoir 160, an array of analysis sites 174 is present. Thermal sensors are located directly upstream (as fluid flows from right to left in the figure) from the array 176 and directly downstream from the array 178. Thermal sensors are also located in the primary channel adjacent to where the primary channel originates at the sample reservoir 180 and adjacent to where the primary channel terminates at the outflow reservoir 182. The supplemental channel provides the location for another thermal sensor 184.

When the device is in operation, the thermal sensor 164 located in or near the sample inlet port 162 is used to indicate the arrival of the sample fluid, e.g. the biomolecular fluid, in the local environment of the thermal sensor, as described herein, and thus provides confirmation that the sample fluid has successfully been introduced into the device. The thermal sensor 180 located in the primary channel 166 adjacent to where the primary channel 166 originates at the sample reservoir 160 produces a signal indicating that sample fluid has started to flow from the sample reservoir 160 into the primary channel 166. The thermal sensors 176 in the primary channel 166 just upstream from the array of analysis sites 174 may be used to indicate that the fluid sample is approaching the array 174. Similarly, the thermal sensors 178 in the primary channel 166 just downstream from the array of analysis sites 174 may be used to indicate that the fluid sample has advanced beyond the array 174 and has thus contacted each analysis site. The thermal sensor 184 in the supplemental channel 172 provides confirmation that the fluid contained within the supplemental reservoir 170 has commenced to flow therefrom. The thermal sensor 182 in the primary channel 166 adjacent to where the primary channel 166 terminates at the outflow reservoir 168 indicates when sample fluid arrives near the outflow reservoir 168, which may then indicate that sufficient sample fluid has passed over the array of analysis sites 174 and that the analysis at the analysis sites is completed. The device described in FIG. 3 is illustrative only, and other potential configurations of the device may be used.

Embodiments of the invention provide for the use of a thermal sensor to detect the arrival of the fluid sample at a determined region, such as an analysis site, in the local environment of the thermal sensor. A variety of thermal sensors may be used. Thermistors are thermally-sensitive resistors whose prime function is to detect a predictable and precise change in electrical resistance when subjected to a corresponding change in temperature Negative Temperature Coefficient (NTC) thermistors exhibit a decrease in electrical resistance when subjected to an increase in temperature and Positive Temperature Coefficient (PTC) thermistors exhibit an increase in electrical resistance when subjected to an increase in temperature. A variety of thermistors have been manufactured for over the counter use and application. Thermistors are capable of operating over the temperature range of −100 degrees to over 600 degrees Fahrenheit. Because of their flexibility, thermistors are useful for application to micro-fluidics and temperature measurement and control.

A change in temperature results in a corresponding change in the electrical resistance of the thermistor. This temperature change results from either an external transfer of heat via conduction or radiation from the sample or surrounding environment to the thermistor, or as an internal application of heat due to electrical power dissipation within the device. When a thermistor is operated in “self-heating” mode, the power dissipated in the device is sufficient to raise its temperature above the temperature of the local environment, which in turn more easily detects thermal changes in the conductivity of the local environment. Thermistors are frequently used in “self heating” mode in applications such as fluid level detection, air-flow detection and thermal conductivity materials characterization. This mode is particularly useful in fluid sensing, since a self-heating conductivity sensor dissipates significantly more heat in a fluid or in a moving air stream than it does in still air.

Embodiments of the invention may be designed such that the thermal sensor is exposed directly to the sample. However, it may also be embedded in the material of the device, e.g., in the wall of a channel meant to transport the sample. The thermal sensor may be covered with a thin coating of polymer or other protective material.

Embodiments of the device need to establish a baseline or threshold value of a monitored parameter such as temperature. Ideally this is established during the setup process. Once fluid movement has been initiated, the device continuously monitors for a significant change thereafter. The change level designated as “significant” is designed as a compromise between noise rejection and adequate sensitivity. The actual definition of the “zero- or start-time” may also include an algorithm determined from the time history of the data, i.e., it can be defined ranging from the exact instant that a simple threshold is crossed, to a complex mathematical function based upon a time sequence of data.

In use, a signal is read from a thermal sensor in the absence of the sample or fluid. The fluid sample is then introduced. The sample flows to or past the site of interest in the local environment of the thermal sensor, and the thermal sensor registers the arrival of the sample. The site of interest may include an analysis site for conducting, e.g., an enzymatic assay. Measuring the arrival of fluid at the site of interest thus indicates the zero- or start-time of the reaction to be performed. For detection of fluid presence, these sites may be any of a variety of desired locations along the fluidic pathway. Embodiments of the invention are particularly well-suited to a microfluidic cartridge or platform which provide the user with an assurance that a fluid sample has been introduced and has flowed to the appropriate locations in the platform.

A rate-based assay must measure both an initiation time, and some number of later time points, one of which is the end-point of the assay. Therefore, baseline or threshold value can be established, and then continuously monitored for a significant change thereafter; one such change is the arrival of the fluid sample that initiates the enzyme reaction. Baseline values are frequently established during the device setup process. The threshold is designed as a compromise between noise rejection and adequate sensitivity. The defined zero- or “start-time” can be defined ranging from the exact instant that a simple threshold is crossed, to the value algorithmically determined using a filter based on a time sequence of data.

Embodiments of the invention accomplish this in a variety of ways. In one embodiment, an initial temperature measurement is made at a thermal sensor without the sample present. The arrival of a sample changes causes the thermal sensor to register a new value. These values are then compared.

Another embodiment measures the change in thermal properties (such as thermal conductivity or thermal capacity) in the local environment of a thermal sensor caused by the arrival of a fluid sample. In general this is the operating principle of a class of devices known as “thermal conductivity sensors” or “heat flux sensors”. At least two hardware implementations have been used and are described above. One implementation utilizes a thermal sensor in a “self-heating mode.” In “self-heating mode,” a self-heating thermal sensor may utilize a positive temperature coefficient thermistor placed in or near the flow channel, e.g. located in the wall of the flow channel. An electrical current is run through the thermistor, causing the average temperature of the thermistor to rise above that of the surrounding environment. The temperature can be determined from the electrical resistance, since it is temperature dependent. When fluid flows through the channel, it changes the local thermal conductivity near the thermistor (usually to become higher) and this causes a change in the average temperature of the thermistor. It also changes the thermal capacity, which modifies the thermal dynamic response. These changes give rise to a signal, which can be detected electronically by well-known means, and the arrival of the fluid can thereby be inferred.

A second hardware implementation requires a separate heating element in or near the flow channel, plus a thermal sensor arrangement in close proximity. Passing a current through the element provides heat to the local environment and establishes a local temperature detected by the thermocouple device. This temperature or its dynamic response is altered by the arrival of the fluid or blood in or near the local environment, similar to the previously described implementation, and the event is detected electronically.

The heating element can be operated in a controlled input mode, which may include controlling one or more of the following parameters—applied current, voltage or power—in a prescribed manner. When operating in controlled input mode, fluctuations of the temperature of the thermal sensor are monitored in order to detect the arrival of the fluid.

Alternatively, the heating element can be operated in such a fashion as to control the temperature of the thermal sensor in a prescribed manner. In this mode of operation, the resulting fluctuations in one or more of the input parameters to the heating element (applied current, voltage, and power) can be monitored in order to detect the arrival of the fluid.

In either of the above-described operating modes, the prescribed parameter can be held to a constant value or sequence of values that are held constant during specific phases of operation of the device. The prescribed parameter can also varied as a known function or waveform in time.

The change in the monitored parameters caused by the arrival of the fluid can be calculated in any of a number of ways, using methods well-known in the art of signal processing. The signal processing methods allow the relation of the signal received prior to arrival of the fluid with the signal received upon arrival of the fluid to indicate that the fluid has arrived. For example, and after suitable signal filtering is applied, changes in the monitored value or the rate of change of the value of the signal can be monitored to detect the arrival of the fluid. Additionally, the arrival of fluid will cause a dynamic change in the thermodynamic properties of the local environment, such as thermal conductivity or thermal capacity. When the input parameter is a time varying function this change of thermodynamic properties will cause a phase shift of the measured parameter relative to the controlled parameter. This phase shift can be monitored to detect the arrival of the fluid.

It should also be noted that sensitivity to thermal noise and operating power levels can be reduced in these either of these modes of operation by a suitable choice of time-varying waveforms for the prescribed parameter, together with appropriate and well-known signal processing methods applied to the monitored parameters. However, these potential benefits may come at the cost of slower response time.

Although the above-described embodiments of the present invention have been described in detail, various modifications to the present invention will become apparent to those skilled in the art from the foregoing description and accompanying drawings and will be within the scope of the invention, which is to be limited only by the following claims.

1. A method of detecting the arrival of a biomolecular fluid in the local environment of a thermal sensor, the method comprising:
(a) obtaining a first signal from the thermal sensor prior to arrival of the biomolecular fluid in the local environment;
(b) obtaining a second signal from the thermal sensor as the biomolecular fluid arrives in the local environment; and
(c) relating the first signal with the second signal to detect the arrival of the biomolecular fluid.

2. The method according to claim 1, wherein the thermal sensor is adjacent an analysis site for analyzing the biomolecular fluid.

3. The method according to claim 1, wherein the thermal sensor is part of a microfluidic device.

4. The method according to claim 3, wherein the thermal sensor is adjacent to a sample inlet port.

5. The method according to claim 3, wherein the thermal sensor is adjacent an analysis site for analyzing the biomolecular fluid

6. The method according to claim 5, wherein the analysis site is for measuring a property of the biomolecular fluid selected from the group consisting of measuring an individual analyte or enzyme, and measuring a characteristic of the total fluid sample.

7. The method according to claim 1, wherein the thermal sensor is adjacent a microfluidic channel.

8. The method according to claim 1, wherein the local environment comprises a porous membrane.

9. The method according to claim 1, wherein the local environment comprises a hydrogel or sol-gel.

10. A method as recited in claim 1, wherein said local environment comprises the biomolecular fluid.

11. A method for determining the start time of a rate-based assay, comprising:
(a) obtaining a first signal from a thermal sensor prior to arrival of the biomolecular fluid in the local environment;
(b) obtaining a second signal from the thermal sensor as the biomolecular fluid arrives in the local environment; and
(c) relating the first signal with the second signal to determine the start time of the rate-based assay.

12. The method according to claim 11, wherein the thermal sensor is adjacent the site of the rate-based assay.

13. The method according to claim 11, wherein the rate-based assay measures an individual analyte.

14. The method according to claim 11, wherein the rate-based assay involves one or more enzymatic reactions and measures a characteristic of the biomolecular fluid.

15. The method according to claim 11, wherein the rate-based assay results in measuring at least one property selected from the group consisting of glucose concentration, clotting time, clot lysis, complement dependent lysis, lactate and creatinine kinase.

16. The method according to claim 11, wherein the rate-based assay is performed in a micro-fluidic device.

17. The method according to claim 11, wherein the local environment comprises a semi-permeable membrane or matrix.

18. The method according to claim 11, wherein said site and local environment is part of a hydrogel or sol-gel.

20. A method for detecting a dynamic change in the local environment of a detector element arising from a biomolecular fluid arriving at or near the local environment, comprising:
a. applying a current to the detector element to raise the temperature of the local environment prior to contact with the biomolecular fluid,
b. receiving a first signal from the detector element prior to arrival of the biomolecular fluid;
c. receiving a second signal from the detector element as the sample or fluid arrives at the local environment; and
d. relating the first signal with the second signal to detect a dynamic change in the local environment.

21. The method according to claim 20, wherein the detector element is adjacent an analysis site for analyzing the biomolecular fluid.

22. The method according to claim 20, wherein the detector element is part of a microfluidic device.

23. The method according to claim 22, wherein the detector element is adjacent a sample inlet port.

24. The method according to claim 22, wherein the detector element is adjacent an analysis site for analyzing the biomolecular fluid.

25. The method according to claim 20, wherein the detector element is a thermistor operated in self-heating mode.

26. The method according to claim 20, wherein the local environment comprises a semi-permeable membrane or matrix.

27. The method according to claim 20, wherein the local environment comprises a hydrogel or sol-gel.

28. A method for determining the start time of a rate-based assay, comprising:
a. Applying a current to a detector element having a local environment to raise the temperature of the local environment prior to the arrival of the biomolecular fluid at the local environment,
b. obtaining a first signal from the detector element prior to arrival of the biomolecular fluid at the local environment;
c. obtaining a second signal from the detector element as the biomolecular fluid arrives at the local environment; and
d. relating the first signal with the second signal to determine the start time of the rate-based assay.

29. The method according to claim 28, wherein the rate-based assay involves one or more enzymatic reactions and measures a characteristic of the biomolecular fluid.

30. The method according to claim 28, wherein the rate-based assay measures an individual analyte.

31. The method according to claim 28, wherein the rate-based assay is performed in a micro-fluidic device.

32. The method according to claim 28, wherein the detector element is a thermistor operated in the self-heating mode.
